SEQUENCE LISTING (15 sequences)

SEQ ID NO: 1 - DNA, 26 bp, Artificial
  GLUT1 forward primer
ggggcatgattggctccttctctgtg

SEQ ID NO: 2 - DNA, 26 bp, Artificial
  GLUT2 forward primer
aactttcatttttggtaggcttatca

SEQ ID NO: 3 - DNA, 26 bp, Artificial
  GLUT3 forward primer
tgtggtcctatgccgaatgccctcag

SEQ ID NO: 4 - DNA, 26 bp, Artificial
  GLUT4 forward primer
cctgcagcctggtagaattgggaagc

SEQ ID NO: 5 - DNA, 26 bp, Artificial
  GLUT5 forward primer
cccgagtcaattaaacagctggtcct

SEQ ID NO: 6 - DNA, 26 bp, Artificial
  GLUT8 forward primer
ccctccttcctgtcatgctccctcca

SEQ ID NO: 7 - DNA, 26 bp, Artificial
  GLUT9 forward primer
ccaaaaacagaacctatgcagaaatc

SEQ ID NO: 8 - DNA, 26 bp, Artificial
  GLUT1 reverse primer
aggccgcagtacacaccgatgatgaa

SEQ ID NO: 9 - DNA, 26 bp, Artificial
  GLUT2 reverse primer
cctcaattaaaaagcaaagcaaacta

SEQ ID NO: 10 - DNA, 26 bp, Artificial
  GLUT3 reverse primer
gcaccaagaagggaaagggagactga

SEQ ID NO: 11 - DNA, 26 bp, Artificial
  GLUT4 reverse primer
ttccctccaatcccccttctctagca

SEQ ID NO: 12 - DNA, 26 bp, Artificial
  GLUT5 reverse primer
ggaagacctgttggggccaccgagtt

SEQ ID NO: 13 - DNA, 26 bp, Artificial
  GLUT8 reverse primer
cccgataaggcagccgcgtgagagga

SEQ ID NO: 14 - DNA, 26 bp, Artificial
  GLUT9 reverse primer
aggccttccatttatcttaccatcag

SEQ ID NO: 15 - protein, 35 aa, Homo sapiens
TRAFEGQAHGADRSGKDGVMEMNSIEPAKETTTNV

(19) United States
(12) Patent Application Publication
(10) Pub. No.: US 2006/0003363 A1
(43) Pub. Date: Jan. 5, 2006

(54) GLUT3 transporters expressed in cancer cells

(75) Inventor: Noa Zerangue, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Xenoport, Inc., Santa Clara, CA (US)

(21) Appl. No.: 11/147,110

(22) Filed: Jun. 6, 2005

Related U.S. Application Data
(60) Provisional application No. 60/577,424, filed on Jun. 4, 2004.

(51) Int. Cl. C12Q 1/68; G01N 33/574

(52) U.S. Cl. 435/6; 435/7.23

(57) ABSTRACT

GLUT3 is consistently expressed at high levels in cancer cells. Disclosed herein are assays for determining whether a test material/molecule is a substrate for, and/or is transported by, the GLUT3 transporter, and therefore a candidate substrate for transport into cancer cells. The assays are useful in screening for cytotoxic agents or imaging components used in the treatment or diagnosis of cancer.

### CONTINUITY

This application claims the benefit of U.S. Provisional Application No. 60/577,424, filed Jun. 4, 2004, which is incorporated by reference herein in its entirety.

### TECHNICAL FIELD

The disclosures herein relate to assays and methods of using the same for screening compounds and/or chemical moieties for their ability to be transported into cancer cells.

### BACKGROUND

Cancer remains the second leading cause of death in the developed world, with solid tumors of the lung, colon, breast, prostate, pancreas, ovary and testis accounting for the majority of cancer deaths. Cancer mortality rates for solid tumors have remained largely unchanged despite the many advances in understanding how solid tumors arise, diagnostic screening, and new cancer drugs.

Small molecule chemotherapeutics typically do not result in a cure for solid tumor cancer, but have clinical value in slowing disease progression, and are an important component of cancer therapy due to their efficacy against a broad range of tumor types and their ability to penetrate solid tumors. These drugs target rapidly dividing malignant cells, halting cell proliferation by interfering with DNA replication, cytoskeletal rearrangements, or signaling pathways that promote cell growth. Disruption of cell division not only slows growth but can also kill tumor cells by triggering cell death. Unfortunately, these drugs also kill normal populations of proliferating cells such as those in the immune system and gastrointestinal tract, causing strong deleterious side effects--including organ failure-that can severely limit tolerated doses and compromise effectiveness.

### SUMMARY OF THE CLAIMED INVENTION

Provided herein are methods of screening an agent, conjugate or conjugate moiety for activity useful for treating or diagnosing cancer, comprising providing a cell expressing a GLUT3 transporter, the transporter being situated in the plasma membrane of the cell; contacting the cell with an agent, conjugate or conjugate moiety; and determining whether the agent, conjugate or conjugate moiety passes through the plasma membrane via the GLUT3 transporter, passage through the GLUT3 transporter being useful for treatment or diagnosis of cancer; wherein if the contacting step comprises contacting the cell with the agent, the agent is a cytotoxic agent or an imaging component; if the contacting step comprises contacting the cell with the conjugate, the conjugate comprises an agent that is a cytotoxic agent or an imaging component; or if the contacting step comprises contacting the cells with the conjugate moiety, the method further comprises linking the conjugate moiety to an agent that is a cytotoxic agent or an imaging component. Some methods further comprise contacting the agent, conjugate, or conjugate moiety, with a cancerous cell and determining whether the agent kills or inhibits growth of the cell. In some methods the cell endogenously expresses the GLUT3 transporter or a nucleic acid molecule encoding the GLUT3 transporter has been transfected or injected into the cell. Some methods further comprise administering the agent, conjugate, or conjugate moiety to an animal and measuring the amount of agent, conjugate, or conjugate moiety that is taken up by cancerous cells in the animal. Some methods further comprise administering the agent, conjugate or conjugate moiety to an undiseased animal and determining any toxic effects.

In some methods the cancerous cell is present in an animal. In some methods the cell is a human cancer cell that has not been genetically manipulated. In other methods the cell is an oocyte. In some methods the cell is a human embryonic kidney (HEK) cell.

In some methods the determining step is performed by a competition assay. In other methods the determining is performed by a direct uptake assay. In some methods the determining step determines that the agent, conjugate or conjugate moiety passes through the plasma membrane via the GLUT3 transporter and the method further comprises modifying the agent, conjugate or conjugate moiety; and determining if the modified agent, conjugate or conjugate moiety is transported with a higher Vₘₐₓ by the GLUT3 transporter than the agent, conjugate or conjugate moiety.

In some methods the cytotoxic agent is selected from the group consisting of platinum, nitrosourea, a phoshoramide group that is selectively cytotoxic to brain tumor cells, nitroimidizole, and nitrogen mustard. In some methods the agent, conjugate or conjugate moiety comprises at least one 5 or 6 membered ring. In some methods the agent, conjugate or conjugate moiety is selected from the list consisting of glucose, glucorionic acid, dehydroascorbic acid, glucosamine, and fluorodeoxyglucose.

Some methods further comprise determining that the agent, conjugate or conjugate moiety is transported by at least one efflux transporter. Additional methods further comprise modifying the agent, conjugate or conjugate moiety; establishing that the modified agent, conjugate or conjugate moiety retains GLUT3 substrate activity; and comparing the ratio of GLUT3 substrate activity to the ratio of efflux substrate activity for the agent, conjugate or conjugate moiety and the modified agent, conjugate or conjugate moiety wherein an increased ratio of GLUT3 substrate activity to efflux substrate activity demonstrates that the modification improves the usefulness of the agent, conjugate or conjugate moiety for treatment or diagnosis of cancer. In some methods the efflux substrate activity is determined by conducting an assay selected from the group consisting of an efflux transporter ATPase activity assay; and an efflux transporter competition assay.

Provided herein are conjugates comprising a cytotoxic agent or imaging component which is transported into cancer cells, identified by screening an agent, conjugate or conjugate moiety for activity useful for treating or diagnosing cancer, comprising providing a cell expressing a GLUT3 transporter, the transporter being situated in the plasma membrane of the cell; contacting the cell with an agent, conjugate or conjugate moiety; and determining whether the agent, conjugate or conjugate moiety passes through the plasma membrane via the GLUT3 transporter, passage through the GLUT3 transporter being useful for treatment or diagnosis of cancer; wherein if the contacting step comprises contacting the cell with the agent, the agent is a cytotoxic agent or an imaging component; if the contacting step comprises contacting the cell with the conjugate, the conjugate comprises an agent that is a cytotoxic agent or an imaging component; or if the contacting step comprises contacting the cells with the conjugate moiety, the method further comprises linking the conjugate moiety to an agent that is a cytotoxic agent or an imaging component; and administering an agent, conjugate, or conjugate moiety to an animal and measuring the amount of agent, conjugate, or conjugate moiety that is taken up by cancerous cells in the animal.

Provided herein are pharmaceutical compositions comprising a cytotoxic agent or an imaging component linked to a conjugate moiety to form a conjugate, wherein the conjugate has a higher Vₘₐₓ for GLUT3 than the cytotoxic agent or the imaging component alone. Some pharmaceutical compositions contain at least one conjugate that has at least 5 times the Vₘₐₓ for GLUT3 than the cytotoxic agent or the imaging component alone. Some pharmaceutical compositions contain at least one conjugate that has a lower Vₘₐₓ for an efflux transporter than the cytotoxic agent or the imaging component alone. Some pharmaceutical compositions contain at least one conjugate moiety that has a Vₘₐₓ for GLUT3 that is at least about 1% of the Vₘₐₓ of glucose for GLUT3. Some pharmaceutical compositions contain at least one conjugate that has a Vₘₐₓ for GLUT3 that is at least 5% of the Vₘₐₓ of glucose for GLUT3. Some pharmaceutical compositions contain at least one conjugate moiety that has a Vₘₐₓ for GLUT3 that is at least about 50% of the Vₘₐₓ of glucose for GLUT3.

Provided herein are methods of formulating a conjugate, comprising linking a cytotoxic agent or imaging component to a conjugate moiety to form the conjugate, wherein the conjugate has a greater Vₘₐₓ for a GLUT3 transporter than the cytotoxic agent or imaging component alone; and formulating the conjugate with a pharmaceutical carrier as a pharmaceutical composition.

Provided herein are methods of delivering a conjugate, comprising administering to a patient a pharmaceutical composition comprising a cytotoxic agent or imaging component linked to a conjugate moiety to form the conjugate, wherein the conjugate has a higher Vₘₐₓ for a GLUT3 transporter than the cytotoxic agent or imaging component alone, and wherein the conjugate is transported into cancerous cells of the patient. In some methods the Vₘₐₓ of the conjugate is at least two-fold higher than that of the cytotoxic agent or imaging component alone. In some methods the cytotoxic agent is selected from the group consisting of platinum, nitrosourea, a phosphoramide group selectively cytotoxic to brain tumor cells, nitroimidizole, and nitrogen mustard. In some methods the cancerous cells are present in a solid tumor. Some methods further comprise determining a level of expression of GLUT3 in the cancerous cells in excess of a level in noncancerous cells from the same tissue. In some methods the cytotoxic agent is a nitroimidizole and the method further comprises irradiating the patient to kill cancerous cells that have taken up the conjugate.

Provided herein are methods of screening an agent for pharmacological activity useful for treating cancer, comprising determining whether an agent binds to a GLUT3 transporter; and contacting the agent with a cancerous cell and determining whether the agent kills or inhibits growth of the cell, killing or inhibition of growth indicating the agent has the pharmacological activity. Some methods further comprise contacting a cell expressing a GLUT3 transporter with a substrate of the GLUT3 transporter, and determining whether the agent inhibits uptake of the substrate into the cancerous cell. In some methods the cell is a HEK cell. In some methods the substrate is selected from the group consisting of glucose, glucorionic acid, dehydroascorbic acid, glucosamine, and fluorodeoxyglucose. Some methods further comprise administering the agent to an undiseased animal and determining any toxic effects.

### BRIEF DESCRIPTION OF THE FIGURES

FIG. 1 shows examples of substrates of the GLUT3 transporter.

FIG. 2 shows GLUT3 expression in oocytes and uptake of radiolabeled glucose.

FIG. 3 shows a competition binding assay in oocytes expressing GLUT3.

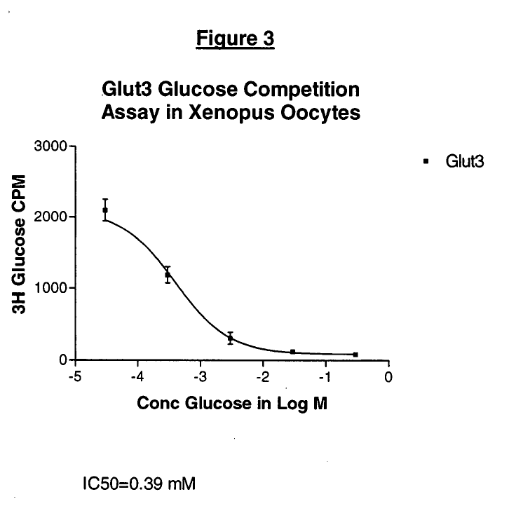

FIG. 4 shows an LCMS assay measuring Glucorionic acid uptake in a HEK cell line expressing GLUT3 and induced with tetracycline.

FIG. 5 shows competition dose response in tetracycline-induced HEK-TREx cells.

FIG. 6 shows an efflux transporter ATPase activity assay using membrane preparations containing the PgP efflux transporter and the PgP substrate verapamil.

FIG. 7 shows an efflux transporter competition assay using the reporter molecule calcein-AM and the PgP substrate verapamil.

### DEFINITIONS

“Transport by passive diffusion” refers to transport of an agent that is not mediated by a specific transporter protein. An agent that is substantially incapable of passive diffusion has a permeability across a standard cell monolayer (e.g., Caco-2 or MDCK cells or an artificial bilayer (PAMPA)) of less than 5×10⁻⁶ cm/sec, and usually less than 1×10⁻⁶ cm/sec in the absence of an efflux mechanism.

A “substrate” of a transport protein is a compound whose uptake into or passage through a cell is facilitated at least in part by a transporter protein.

The term “ligand” of a transporter protein includes compounds that bind to the transporter protein. Some ligands are transported and are thereby also substrates. Some ligands by binding to the transport protein inhibit or antagonize uptake of the substrate or passage of substrate through a cell by the transport protein. Some ligands by binding to the transport protein promote or agonize uptake or passage of the compound by the transport protein or another transport protein. For example, binding of a ligand to one transport protein can promote uptake of a substrate by a second transport protein in proximity with the first transport protein.

The term “agent” is used to describe a compound that has or may have a pharmacological activity. Agents include compounds that are known drugs, compounds for which pharmacological activity has been identified but which are undergoing further therapeutic evaluation, and compounds that are members of collections and libraries that are to be screened for a pharmacological activity.

An agent is “orally active” if it can exert a pharmacological activity when administered via an oral route.

A “conjugate” refers to a compound comprising an agent and a chemical moiety bound thereto, which moiety by itself or in combination with the agent renders the conjugate a substrate for transport, for example rendering the conjugate to be a substrate for a transport protein. The chemical moiety may or may not be subject to cleavage from the agent upon uptake and metabolism of the conjugate in the patient's body. In other words, the moiety may be cleavably bound to the agent or non-cleavably bound to the agent. The bond can be a direct (i.e., covalent) bond or the bond can be through a linker. In cases where the bond/linker is cleavable by metabolic processes, the agent, or a further metabolite of the agent, is the therapeutic entity. In cases where the bond/linker is not cleavable by metabolic processes, the conjugate is the therapeutic entity. The conjugate can comprise a prodrug having a metabolically cleavable moiety, where the conjugate itself does not have pharmacological activity but the agent to which the moiety is cleavably bound does have pharmacological activity. Typically, the moiety facilitates therapeutic use of the agent by promoting uptake of the conjugate via a transporter. Thus, for example, a conjugate comprising an agent and a conjugate moiety may have a Vₘₐₓ for a GLUT3 transporter that is at least 2, 5, 10, 20, 50 or 100-fold higher than that of the agent alone. A conjugate moiety can itself be a substrate for a transporter or can become a substrate when linked to the agent (e.g., valacyclovir, an L-valine ester prodrug of the antiviral drug acyclovir). Thus, a conjugate formed from an agent and a conjugate moiety can have higher uptake activity than either the agent or the moiety alone.

A “cancerous cell” is a cell that has lost or partially lost the ability to control cell division. A cancerous cell can be a cell line such as HeLa, MOLT4, and others, and can also be a cell obtained from a patient. A cancerous cell from a patient can be from a solid tumor (such as a tumor of the colon) or from a non-solid tissue such as blood (e.g, leukemia). A cancerous cell can be isolated from a human or animal, such as cells obtained from a tissue biopsy. Alternatively, a cancer cell can be present in a human or animal. Cancerous cells are also referred to as tumor cells.

Malignant cancers are those that invade surrounding tissues and metastasize (spread) to other body sites via the blood and lymphatic circulations. Metastasized cancers usually remain the same type of cell as the initial site of cancer development; for example, if breast cancer metastasizes to a lung, the cancer in the lung consists of breast cells. Benign cancers do not invade other tissues or spread, have a slower growth rate than malignant cancers, and in most cases are not fatal.

The term “treating” includes achieving a therapeutic benefit and/or a prophylactic benefit.

A cell has been “genetically manipulated” when its genome sequence has been altered by a practitioner. A cell can be genetically manipulated through the introduction of a nucleic acid into the cell. Alternatively, a cell can be genetically manipulated through exposure to molecules that mutate DNA sequences, such as nitrosoguanidine.

A “pharmacological” activity means that an agent exhibits an activity in a screening system that indicates that the agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

Vₘₐₓ and Kₘ of a compound for a transporter are defined in accordance with convention. Vₘₐₓ is the number of molecules of compound transported per second at saturating concentration of the compound. Kₘ is the concentration of the compound at which the compound is transported at half of Vₘₐₓ. When the goal is to transport an agent, conjugate or conjugate moiety into a cancer cell, a high Vₘₐₓ for an influx transporter such as GLUT3 is generally desirable. Likewise for the same goal, a low value of Kₘ is typically desirable for transport of a compound present at low blood concentrations. In some instances a high value of Kₘ is acceptable for the transport of compounds present at high concentrations in the blood. For these reasons, the intrinsic capacity of a compound to be transported by a particular transporter is usually expressed as the ratio Vₘₐₓ of the compound/Vₘₐₓ of a reference compound known to be a substrate for the transporter. Vₘₐₓ is affected both by the intrinsic turnover rate of a transporter (molecules/transporter protein) and transporter density in the plasma membrane, which depends on expression level.

“EC50”, or “effective concentration 50”, is a measurement of the substrate concentration that results in a turnover rate 50% of the maximal turnover rate for the substrate (0.5 Vₘₐₓ).

“Sustained release” refers to release of a therapeutic or prophylactic amount of a drug or an active metabolite thereof over a period of time that is longer than a conventional formulation of the drug. For oral formulations, the term “sustained release” typically means release of the drug within the GI tract lumen over a period of from about 2 to about 30 hours, more typically over a period of about 4 to about 24 hours. Sustained release formulations achieve therapeutically effective concentrations of the drug in the systemic blood circulation over a prolonged period of time relative to that achieved by oral administration of a conventional formulation of the drug. “Delayed release” refers to release of the drug or an active metabolite thereof into the gastrointestinal lumen after a delay time period, typically a delay of about 1 to about 12 hours, relative to that achieved by oral administration of a conventional formulation of the drug.

The phrase “specifically binds” when referring to a substrate or ligand of a GLUT3 transporter refers to a specific interaction between a substrate or ligand and the GLUT3 transporter which determines the presence of GLUT3 in a heterogeneous mixture of proteins and other biological molecules. Thus, the substrate or ligand binds preferentially with a GLUT3 transporter and does not bind in a significant amount to most or any other proteins present in a biological sample. A substrate or ligand that specifically binds to a GLUT3 transporter often has an association constant of 10×10⁴ M⁻¹, 10⁵ M⁻¹, 10⁶ M⁻¹ or 10⁷ M⁻¹, preferably 10⁸ M⁻¹ to 109 M⁻¹ or higher. However, some substrates or ligands of GLUT3 transporters have much lower affinities and yet the binding is still specific. Substrates of GLUT3 can specifically bind to GLUT3 and other proteins such as efflux transporters without specifically binding to other proteins.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope hereof, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLASTN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### DETAILED DESCRIPTION

I. General

GLUT3 is shown herein to be expressed at high levels in cancer cells. This finding can be used to generate or isolate conjugates and agents having cytotoxic or imaging activity useful for treatment, prophylaxis or diagnosis of cancer. The invention provides methods of identifying agents, conjugates or conjugate moieties that are substrates for GLUT3. Agents or conjugates having inherent cytotoxic activity can be screened to determine whether they are substrates for GLUT3. Alternatively, a conjugate moiety lacking such activity can be screened, and linked to a cytotoxic agent after screening. Agents or conjugates that both have cytotoxic activity and are substrates for GLUT3 are preferentially transported into cancer cells via GLUT3 transporters after administration to a patient. Such an agent or conjugate by itself or in combination with another agent is effective in treatment or prophylaxis of cancer. An analogous approach is used for imaging tumors. Agents and conjugates that have an imaging component and are substrates for GLUT3 are preferentially transported into cancer cells via GLUT3 transporters. The imaging component is then detected by various methods such as detecting radioactive decay of the imaging component. The agents and conjugates can be used to image tumors overexpressing the GLUT3 transporter. Optionally, the agents or conjugates have inherent affinity for, or are provided with a conjugate moiety that confers affinity for, a particular antigen or cell type contained within a tumor.

II. GLUT3 transporter

The family of facilitated glucose transporters (GLUTs) contains at least 14 members in humans (SLC1A1-14, GLUT3-14). GLUT transporters have 12 putative transmembrane domains, with both the amino and carboxy termini located on the cytoplasmic side. Various GLUT transporters have been demonstrated to transport a variety of sugars (glucose, 2-deoxyglucose, galactose, fructose, inositol) and sugar analogs (dehydroascorbate, glucosamine, and fluorodeoxyglucose). Transport is bidirectional, allowing transport either into or out of the cell depending on the substrate gradients. Because there is no net charge movement, transport does not depend on the membrane potential.

It is now shown that GLUT3 is highly expressed in cancer cells. GLUT3 is expressed at a level more than 1000-fold higher than some other GLUT family transporters with similar substrate specificity, as shown in Table 3. It is desirable to generate agents, conjugates, and conjugate moieties that have activity for GLUT3 for transport into cancer cells due to this high expression level. The GenBank accession number for human GLUT3 is NM_{—}006931 (incorporated by reference). Unless otherwise apparent from the context, reference to a transporter includes the amino acid sequence described in or encoded by the GenBank reference number NM_{—}00693 1, and, allelic, cognate and induced variants and fragments thereof retaining essentially the same transporter activity. Usually such variants show at least 90% sequence identity to the exemplary GenBank nucleic acid or amino acid sequence.

III. Methods of Screening to Identify Substrates

Agents known or suspected to have a cytotoxic activity or to comprise an imaging component can be screened directly for their capacity to act as substrates of GLUT3. Alternatively, conjugate moieties can be screened as substrates, and the conjugate moieties are then linked to a cytotoxic agent or imaging component. In such methods, the conjugate moieties can optionally be linked to a cytotoxic agent or imaging component, or other molecule during the screening process. If another molecule is used in place of a cytotoxic agent or imaging component, the molecule can be chosen to resemble the structure of a cytotoxic agent or imaging component ultimately intended to be linked to the conjugate moiety for therapeutic use. Alternatively, a conjugate moiety can be screened for a substrate activity alone and linked to a cytotoxic agent or imaging component after screening.

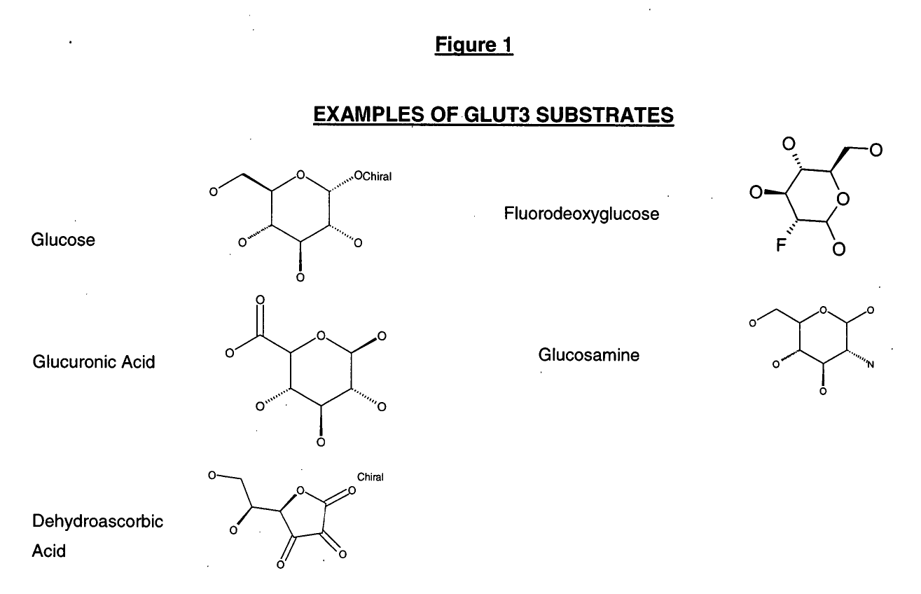

Preferred substrates for GLUT3 are compounds containing 5 and 6 membered rings. Preferred substrates have alcohol groups attached to several of the positions on the ring. Substrates of GLUT3 are typically sugars and vitamins. Table 1 lists examples of substrates of GLUT3. The structures of each compound listed in Table 1 are depicted in FIG. 1.

TABLE 1
SUBSTRATES

Glucose
Glucuronic acid
Dehydroascorbic Acid
Glucosamine
Fluorodeoxyglucose

Glucose, glucorionic acid, dehydroascorbic acid, glucosamine, and fluorodeoxyglucose are examples of GLUT3 substrates that are candidates for conjugation to therapeutic neuropharmaceutical agents, cytotoxic neuropharmaceutical agents and imaging components.

In some screening methods, the cells are transfected with DNA encoding the GLUT3 transporter. HEK (human embryonic kidney) and CHO (Chinese hamster ovary) cells, for example, are suitable for transfection. Oocytes can be injected with GLUT3 cRNA to express GLUT3 transporter. In some methods, the only transporter expressed by the cells is the GLUT3 transporter. In other methods, cells express GLUT3 in combination with other transporters. In still other methods, agents, conjugate moieties or conjugates are screened on different cells expressing different transporters. Agents, conjugate moieties or conjugates can be screened either for specificity for the GLUT3 transporter or for transport into cells endogenously expressing a plurality of transporters. Cells lacking GLUT3 transporters can be used as negative controls in such experiments.

In some methods, cells endogenously expressing the GLUT3 transporter are used. Certain cancer cell lines, for example, endogenously express the GLUT3 transporter. Cells from certain tumor types also express the GLUT3 transporter. Agents, conjugate moieties or conjugates can be screened for transport into cells of cancer cell lines or primary cultures of cancer cells.

In some methods, the ability of an agent, conjugate or conjugate moiety to specifically bind to a GLUT3 transporter is tested. A known substrate of the GLUT3 transporter and the agent, conjugate or conjugate moiety are added to cells expressing the GLUT3 transporter. The amount or rate of transport of the substrate in the presence of the agent, conjugate or conjugate moiety is compared to the amount or rate of transport of the agent, conjugate or conjugate moiety in the absence of the test compound. If the amount or rate of transport of the substrate is decreased by the presence of the agent, conjugate or conjugate moiety, the agent, conjugate or conjugate moiety binds the GLUT3 transporter. Agents, conjugates or conjugate moieties that bind the GLUT3 transporter can be further analyzed to determine if they are transported by the GLUT3 transporter or only adhere to the exterior of the transporter. Agents, conjugates or conjugate moieties that are transported by the GLUT3 transporter in cultured cell lines can be further tested to determine if they are transported by cancer cells within their natural environment within a tumor. Agents and conjugates having cytotoxic activity and that that are transported by the GLUT3 transporter can be used to form pharmaceutical compositions. Conjugate moieties that are transported by the GLUT3 transporter can be linked to a cytotoxic agent or an imaging component.

Transport of a compound into a cell can be detected by detecting a signal from within a cell from any of a variety of reporters. The reporter can be as simple as a label such as a fluorophore, a chromophore, or a radioisotope. Confocal imaging can also be used to detect internalization of a label as it provides sufficient spatial resolution to distinguish between fluorescence on a cell surface and fluorescence within a cell; alternatively, confocal imaging can be used to track the movement of compounds over time. In another approach, transport of a compound is detected using a reporter that is a substrate for an enzyme expressed within a cell. Once the compound is transported into the cell, the substrate is metabolized by the enzyme and generates an optical signal that can be detected. Light emission can be monitored by commercial PMT-based instruments or by CCD-based imaging systems. In addition, assay methods utilizing liquid chromatography-mass spectroscopy (LC-MS-MS) detection of the transported compounds or electrophysiological signals indicative of transport activity are also employed. Mass spectroscopy is a powerful tool because it allows detection of very low concentrations of almost any compound, especially molecules for which a radiolabeled version is not available. It can also be used to distinguish substrates from nontransported ligands.

In some methods, multiple agents, conjugates or conjugate moieties are screened simultaneously and the identity of each agent, conjugate or conjugate moiety is tracked using tags linked to the agents, conjugates or conjugate moieties. In some methods, a preliminary step is performed to determine binding of an agent, conjugate or conjugate moiety to a transporter. Although not all agents, conjugates or conjugate moieties that bind to a transporter are substrates of the transporter, observation of binding is an indication that allows one to reduce the number of candidates from an initial repertoire. In some methods, the transport rate of an agent, conjugate or conjugate moiety is tested in comparison with the transport rate of a reference substrate for that transporter. For example, glucose, a natural substrate of GLUT3, can be used as a reference. The comparison can be performed in separate parallel assays in which an agent, conjugate or conjugate moiety under test and the reference substrate are compared for uptake on separate samples of the same cells. Alternatively, the comparison can be performed in a competition format in which an agent, conjugate or conjugate moiety under test and the reference substrate are applied to the same cells. Typically, the agent, conjugate or conjugate moiety and the reference substrate are differentially labeled in such assays.

In comparative assays, the Vₘₐₓ of an agent, conjugate or conjugate moiety tested can be compared with that of a reference substrate. If an agent, conjugate moiety or conjugate has a Vₘₐₓ of at least 1%, 5%, 10%, 20%, and most preferably at least 50% of the reference substrate for the GLUT3 transporter, then the agent, conjugate moiety or conjugate is also a substrate for the GLUT3 transporter. If transport of the agent, conjugate moiety or conjugate into a cancer cell is desired, a higher Vₘₐₓ of the agent, conjugate moiety or conjugate relative to that of the reference substrate is preferred. Therefore, agents, conjugate moieties or conjugates having Vₘₐₓ's of at least 1%, 5%, 10%, 20%, 50%, 100%, 150% or 200% (i.e., two-fold) of the Vₘₐₓ of a reference substrate (e.g., glucose) for the transporter are screened in some methods. The components to which conjugate moieties are linked can by themselves show little or no detectable substrate activity for the transporter (e.g., Vₘₐₓ relative to that of a reference substrate of less than 0.1% or 1%). Preferred agents, conjugates or conjugate moieties have a Vₘₐₓ for GLUT3 that is at least 5% of the Vₘₐₓ for GLUT3 of glucose. Preferred conjugates comprising a cytotoxic agent or imaging component linked to a conjugate moiety preferably have a greater Vₘₐₓ for GLUT3 than the cytotoxic agent or imaging component alone.

Having determined that an agent, conjugate or conjugate moiety is a substrate for GLUT3, a further screen can be performed to determine its cytotoxic activity against cancer cells. If the agent, conjugate or conjugate moiety does not have inherent cytotoxic activity, it is first linked to another chemical component having such cytotoxic properties. The agent, conjugate or conjugate moiety is then contacted with cells expressing GLUT3. The contacting can be performed either on a population of cells in vitro, or the cancer cells of a test animal via administration of the agent, conjugate or conjugate moiety to a test animal. The cytotoxic activity of the agent, conjugate or conjugate moiety is then determined from established protocols for that particular form of cancer. Optionally, the effect of the agent, conjugate or conjugate moiety can be compared with a placebo.

A further screen can be performed to determine toxicity of the agent, conjugate, or conjugate moiety to normal cells. The agent, conjugate or conjugate moiety is administered to a laboratory animal that is preferably in an undiseased state. Various tissues of the animal, such as liver, kidney, heart and brain are then examined for signs of pathology. Cells in the animal can also be analyzed for uptake of the agent, conjugate, or conjugate moiety.

IV. Iterative Modification and Testing of GLUT3 Substrates

Having determined that an agent, conjugate or conjugate moiety is a substrate for GLUT3, the agent, conjugate or conjugate moiety can be modified to improve its properties as a substrate. The modified agent, conjugate or conjugate moiety is then tested for transport by GLUT3. Modified agents, conjugates or conjugate moieties that are transported by GLUT3 at a higher Vₘₐₓ compared to the unmodified agent, conjugate or conjugate moiety are preferred. The process of modifying agents, conjugates or conjugate moieties and testing for transport by GLUT3 can be repeated until a desired level of transport is reached.

Agents, conjugates or conjugate moieties that are substrates of GLUT3 can also be modified for decreased capacity to be transported out of cells by efflux transporters. An agent, conjugate or conjugate moiety transported by GLUT3 is assayed to determine whether it is also a substrate for one or more efflux transporters. If the agent, conjugate or conjugate moiety is transported by an efflux transporter, the agent, conjugate or conjugate moiety is modified and tested for both reduced transport by an efflux transporter and retention of GLUT3 substrate activity.

In some instances, the specific efflux transporter responsible for transporting an agent, conjugate or conjugate moiety is known. The agent, conjugate or conjugate moiety is modified, preferably by addition of a chemical group that differs in chemical characteristics from other known substrates of the efflux transporter. The modified agent, conjugate or conjugate moiety is then tested for retained capacity to be transported by GLUT3 and a diminished capacity to be transported by an efflux transporter. It is not necessary that the modified agent, conjugate or conjugate moiety retain the same kinetic properties of GLUT3 transporter substrate as the unmodified agent, conjugate or conjugate moiety as long as some GLUT3 substrate activity is retained. Examples of efflux transporters are the P-glycoprotein (PgP), multidrug resistance protein (MRP1), and breast cancer resistance protein (BCRP). Preferred agents, conjugates or conjugate moieties have a GLUT3 transport:efflux transport ratio of at least 1.1: 1.0, more preferably, 2.0:1.0, and more preferably 5.0:1.0 and more preferably 10.0:1.0 or higher at a given concentration of agent, conjugate or conjugate moiety.

Efflux transporter activity can be measured in several ways. First, functional assays can be performed in which interaction of compounds with efflux transporters is measured by stimulation of efflux transporter ATPase activity in cellular membrane fragments or vesicles. Second, competition assays can be performed in which test compounds compete with known efflux substrates in whole cells. Other assays besides these two can also be used to directly or indirectly measure the efflux substrate characteristics of a test compound.

The efflux transporter ATPase assay is based on the fact that most efflux substrates increase the ATPase activity of efflux transporters upon binding. In one type of assay, Baculovirus membrane fragments or vesicles containing an efflux transporter such as PgP, as well as control membrane fragments or vesicles not containing the efflux transporter, are either prepared or obtained from commercial suppliers. The ATPase activity of the membrane fragments or vesicles is measured in the presence of various concentrations of the test compound. An agent, conjugate, or conjugate moiety that is transported by GLUT3 is added to the ATPase assay reaction and the amount of ATPase activity is measured at various concentrations of agent, conjugate, or conjugate moiety. Parallel experiments are performed in which ATPase activity is measured under addition of the same concentrations of modified agent, conjugate, or conjugate moiety that retain GLUT3 substrate activity. Reduced ATPase activity caused by the modified agent, conjugate, or conjugate moiety compared to the unmodified agent, conjugate, or conjugate moiety indicates that the modified agent, conjugate, or conjugate moiety is a better candidate for retention in cancer cells.

In the competition assay, the test compound is assayed for competition with a known efflux substrate. For example, calcein-AM is a non-fluorescent compound that is a substrate of PgP and MRP1. Calcein-AM is initially loaded into the cells, for example, by transport by passive diffusion. Cells expressing these efflux transporters actively efflux nearly all of the calcein-AM that is present in the cells. However, when other efflux transporter substrates are present, these other substrates compete with calcein-AM for efflux, resulting in more calcein-AM accumulating inside the cells. Intracellular esterases convert the non-fluorescent calcein-AM to fluorescent calcein which can be measured spectrophotometrically. An agent, conjugate, or conjugate moiety that is transported by GLUT3 is loaded into efflux transporter-containing cells by either GLUT3 transport or passive diffusion. Calcein-AM is also loaded into the cells by active transport or transport by passive diffusion. Accumulation of calcein-AM is measured and compared to the amount of accumulation in the absence of the agent, conjugate, or conjugate moiety. Parallel experiments are performed in which a modified agent, conjugate, or conjugate moiety that is transported by GLUT3 is loaded into the cells. Accumulation of calcein-AM is measured and compared to the amount of accumulation in the absence of the modified agent, conjugate, or conjugate moiety. Decreased calcein-AM accumulation inside the cells caused by the presence of a modified agent, conjugate, or conjugate moiety compared to calcein-AM accumulation in the presence of unmodified agent, conjugate, or conjugate moiety indicates that the modified agent, conjugate, or conjugate moiety is a better candidate for retention inside cancer cells.

The cells used for competition assays can be cells that either express a high endogenous level of the efflux transporter of interest or are transformed with an expression vector containing the efflux transporter gene. Suitable cell lines for efflux assays are, for example, HEK and MDCK cell lines into which the PgP gene has been transfected, or MES-SA/Dx5 uterine sarcoma cells grown in the presence of 500 nM doxorubicin, which express a high endogenous level of PgP. These cells can optionally be transfected with the GLUT3 transporter gene. Preferred cells express both one or more efflux transporter genes such as PgP and the GLUT3 gene, either endogenously or through transfection of expression vectors.

An additional screen can be performed to determine whether agents, conjugates or conjugate moieties have substantial capacity for passive diffusion into cancer cells. Such an assay can be performed using cells lacking GLUT3 transporters. That is, the agents, conjugates or conjugate moieties are exposed to cells that lack GLUT3 transporters, and the amount of agents, conjugates or conjugate moieties that are present inside the cell is measured.

V. Agents, Cytotoxic Agents, Imaging Components

The agents, conjugate or conjugate moieties to be screened as substrates of GLUT3 are usually vitamins and sugar compounds. Agents can be obtained from natural sources such as, e.g., marine microorganisms, algae, plants, and fungi. Alternatively, agents can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, heterocyclic and other organic compounds, lipids, glucocorticoids, antibiotics, peptides, sugars, carbohydrates, and chimeric molecules.

Typically if an agent is being screened, the agent is known or suspected to have an inherent cytotoxic or imaging activity. If a conjugate is being screened, the conjugate usually comprises an agent being screened for substrate activity linked to a known cytotoxic agent or imaging component. If a conjugate moiety is being screened, the conjugate moiety typically lacks cytotoxic or imaging activity and this is added after screening.

Suitable cytotoxic components for incorporation into conjugates or linkage to conjugate moieties after screening include platinum, nitrosourea, nitrogen mustard, a phosphoramide group that is only cytotoxic to cancer cells when taken up by a transporter. Radiosensitizers, such as nitroimidizoles, can also be used. The choice of imaging component depends on the means of detection. For example, a fluorescent imaging component is suitable for optical detection; A paramagnetic imaging component is suitable for tomographic detection without surgical intervention. Radioactive labels can also be detected using PET or SPECT.

The agents, conjugates or conjugate moieties to be screened optionally linked to a cytotoxic agent or imaging component if not inherently present are preferably small molecules having molecular weights of less than 1000 Da and preferably less than 500 Da.

VI. Linkage of Cytotoxic or Imaging Components to Substrates

Conjugates can be prepared by either by direct conjugation of a cytotoxic agent or imaging component to a substrate for GLUT3 with a covalent bond (optionally cleavable in vivo), or by covalently coupling a difunctionalized linker precursor with the cytotoxic or imaging component and substrate. The linker precursor is selected to contain at least one reactive functionality that is complementary to at least one reactive functionality on the cytotoxic or imaging component and at least one reactive functionality on the substrate. Optionally, the linker is cleavable. Suitable complementary reactive groups are well known in the art as illustrated below:

TABLE 2
COMPLEMENTARY BINDING CHEMISTRIES

| First Reactive Group | Second Reactive Group | Linkage |
|---|---|---|
| hydroxyl | carboxylic acid | ester |
| hydroxyl | haloformate | carbonate |
| thiol | carboxylic acid | thioester |
| thiol | haloformate | thiocarbonate |
| amine | carboxylic acid | amide |
| hydroxyl | isocyanate | carbamate |
| amine | haloformate | carbamate |
| amine | isocyanate | urea |
| carboxylic acid | carboxylic acid | anhydride |
| hydroxyl | phosphorus acid | phosphonate or |
| | | phosphate ester |

VII. Pharmaceutical Compositions

The above screening processes result several entities to be incorporated into pharmaceutical compositions. These entities include agents that are both substrates for GLUT3 and have inherent cytotoxic or imaging activity. The entities also include conjugates in which a cytotoxic agent or imaging component is linked to a substrate for GLUT3.

The above entities are combined with pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington's pharmaceutical Sciences, Mace Publishing Company, Philadelphia, Pa., 17th ed. (1985); for a brief review of methods for drug delivery, see, Langer, Science 249:1527-1533 (1990); each of these references is incorporated by reference in its entirety).

Pharmaceutical composition can be administered administered topically, orally, intranasally, intradermally, subcutaneously, intrathecally, intramuscularly, topically, intravenously, or injected directly to a site of cancerous tissue. For parenteral administration, the compounds disclosed herein can be administered as injectable dosages of a solution or suspension of the compound in a physiologically acceptable diluent with a pharmaceutical carrier which can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymers thereof for enhanced adjuvant effect, as discussed above (see Langer, Science 249, 1527 (1990) and Hanes, Advanced Drug Delivery Reviews 28, 97-119 (1997). The pharmaceutical compositions disclosed herein can be administered in the form of a depot injection or implant preparation which can be formulated in such a manner as to permit a sustained or pulsatile release of the active ingredient.

Pharmaceutical compositions for oral administration can be in the form of e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. Preserving agents such as methyl- and propylhydroxy-benzoates; sweetening agents; and flavoring agents can also be included. Depending on the formulation, compositions can provide quick, sustained or delayed release of the active ingredient after administration to the patient. Polymeric materials can be used for oral sustained release delivery (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105). Sustained release can be achieved by encapsulating conjugates within a capsule, or within slow-dissolving polymers. Preferred polymers include sodium carboxymethylcellulose, hydroxypropylcellulose, hydroxypropylmethylcellulose and hydroxyethylcellulose (most preferred, hydroxypropylmethylcellulose). Other preferred cellulose ethers have been described (Alderman, Int. J. Pharm. Tech. & Prod. Mfr., 1984, 5(3) 1-9). Factors affecting drug release have been described in the art (Bamba et al., Int. J. Pharm., 1979, 2, 307). For administration by inhalation, the compounds for use according to the disclosures herein are conveniently delivered in the form of an aerosol spray preparation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Effective dosage amounts and regimes (amount and frequency of administration) of the pharmaceutical compositions are readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The components of pharmaceutical compositions are preferably of high purity and are substantially free of potentially harmful contaminants (e.g., at least National Food (NF) grade, generally at least analytical grade, and more typically at least pharmaceutical grade).

To the extent that a given compound must be synthesized prior to use, the resulting product is typically substantially free of any potentially toxic agents, particularly any endotoxins, which may be present during the synthesis or purification process. Compositions are usually made under GMP conditions. Compositions for parenteral administration are usually sterile and substantially isotonic.

VIII. Methods of Treatment

The pharmaceutical compositions disclosed herein are used in methods of treating cancer. Examples of tumors amenable to treatment are cancers of the bladder, brain, breast, colon, esophagus, kidney, leukemia, liver, lung, oral cavity, ovary, pancreas, prostate, skin, stomach and uterus. The compositions are particularly useful for treating solid tumors, such as sarcoma, lymphomas and carcinomas. Preferred cancers for treatment are those shown in Table 3 in which expression of GLUT3 is higher in the cancer than in normal cells from the tissue. Examples of these cancers include brain cancers, such as astrocytoma, glioblastoma multiforme, malignant ependymana, and medullablastoma. Breast cancers amenable to treatment include infiltrating ductal adenocarcinoma, ductal adenocarcinoma, and lobular adenocarcinoma. Lung cancers amenable to treatment include squamous cell carcinoma and epidermoid carcinoma. Colon cancers amenable to treatment include colon adenocarcinoma, medullary carcinoma, and mucinous carcinoma. Prostate cancers amenable to treatment include prostate sarcoma. Incorporation of other isotopes such as boron (¹⁰B) allows boron neutron capture therapies (BNCT) in which low-energy neutron irradiation is used to induce boron decay and release of higher energy particles that are toxic to cells. An advantage this and similar approaches relative to existing chemotherapy approaches is that release of particles from decaying isotopes could kill neighboring cells as well, and provide more complete tumor killing in poorly vascularized solid tumors. Another advantage of these approaches is that tumors in highly radiation sensitive tissues (liver, pancreas) can be targeted.

In prophylactic applications, pharmaceutical compositions are administered to a patient susceptible to, or otherwise at risk of, cancer in an amount and frequency sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, pharmaceutical compositions are administered to a patient suspected of, or already suffering from such a disease in an amount and frequency sufficient to cure, or at least partially arrest, the symptoms of the disease (biochemical, histologic and/or behavioral), including its complications and intermediate pathological phenotypes in development of the disease. An amount of pharmaceutical composition sufficient to achieve at least one of the above objects is referred to as an effective amount, and a combination of amount and frequency sufficient to achieve at least one of the above objects is referred to as an effective regime.

Optionally, administration of a pharmaceutical composition is combined with administration of a second chemotherapeutic agent or radiation. For example, in some methods, the pharmaceutical composition comprises a substrate of GLUT3 linked to a cytotoxic component that renders a cell susceptible to radiation damage.

IX. Methods of Imaging

As discussed above, the invention provides conjugates comprising a conjugate moiety, which is a substrate of GLUT3, linked to an imaging component, as well as agents that are substrates for GLUT3 and have an inherent imaging activity. Optionally, the agents also have inherent affinity for a particular antigen or cell type found in cancer cells, or the conjugate is provided with an additional conjugate moiety having such affinity. The additional moiety is referred to as a targeting moiety. The targeting moiety can be an antibody or fragment thereof, or any other molecule that specifically binds to a desired antigen or cell type. The invention further provides pharmaceutical compositions comprising all of these entities. These pharmaceutical compositions can be used for in vivo imaging. The compositions are administered to a patient and preferentially taken up by cancer cells expressing GLUT3 in the patient. The imaging activity is then detected. In some methods, the imaging component is also a cytotoxic agent. For example many radioisotopes are suitable for both imaging and tumor cytotoxic activity. In such cases, methods of imaging and methods of treatment can be combined. Currently used diagnostic imaging techniques include positron emission tomography (PET), magnetic resonance imaging (MRI), and computed tomography (CT). Transported imaging components provide information about, for example, the presence and/or size of a tumor.

As can be appreciated from the disclosure above, the present invention has a wide variety of applications. Accordingly, the following examples are offered by way of illustration, not by way of limitation.

### EXAMPLES

### Example 1

Quantitative PCR Detection of GLUT Transporters Expression in Tumor Cells

To measure the level of GLUT transporter expression in human tumors, quantitative PCR was performed on human tumor mRNA purchased from Ardais Corporation. For comparison with normal colon, human colon mucosal tissue was obtained from endoscopy procedures. Table 3 shows high levels of GLUT3 mRNA in human tumors.

Intestinal biopsy samples were obtained, with patient consent, from routine endoscopies or colonoscopies. Biopsies were taken from healthy sites by Radial Jaw 3 single use biopsy forceps (Boston Scientific) within the endoscope working channel. Each sample was approximately 3 mm in size. Samples were placed in numbered cryovials and snap frozen in liquid nitrogen. Vials were stored at −80° C. Biopsies were taken from up to three sites from a single patient.

Total RNA was isolated from all samples using the RNeasy RNA Isolation Kit (Qiagen). 1500 ul RLT Lysis Buffer+1% μ-me was added to each biopsy. Samples were homogenized with a Powergen 125 Tissue Homogenizer (Fischer Scientific). Lysates were run though a Qiashredder column prior to RNA isolation (Qiagen). Total RNA was isolated by following the RNeasy RNA Isolation manual. RNA was quantified and run on an agarose gel to ensure RNA integrity.

To synthesize single-stranded cDNA, one microgram of DNAseI treated total RNA (Invitrogen) was used as template per oligo dT primed Thermoscript RT reaction (Invitrogen). Following the completion of cDNA synthesis, RNA template was destroyed by RNAse H addition for 20 minutes at 37° C. To quantify mRNA, single-stranded cDNA was amplified using GLUT transporter specific primers in an MJ Research real-time PCR instrument using SYBR green fluorescent detection. Sample data was normalized using the mRNA abundance of GAPDH, and data shown in Table 3 indicates number of mRNA transcripts in the quantitative PCR reaction.

TABLE 3
GLUT family mRNA Expression in
human stage 2 adenocarcinomas

| | mRNA level- stage 2 adenocarcinoma | | | | | |
|---|---|---|---|---|---|---|
| | Breast | Lung | Colon | Ovary | Tumor:Normal Ratio | |
| | n = 11 | n = 11 | n = 15 | n = 4 | Colon | Ovary |
| GLUT1 | 11717 | 19364 | 10774 | 12009 | 1.6 | 9.7 |
| GLUT2 | 7 | 0 | 3 | 232 | 0.8 | 1.0 |
| GLUT3 | 9695 | 24302 | 8391 | 25032 | 0.7 | 1.8 |
| GLUT4 | 144 | 147 | 80 | 29 | 12.5 | 0.3 |
| GLUT5 | 862 | 2951 | 1097 | 3148 | 0.3 | 3.0 |
| GLUT8 | 1019 | 260 | 50 | 1121 | 0.4 | 0.4 |
| GLUT9 | 23 | 5 | 0 | 696 | 0.2 | 0.3 |

TABLE 4

Primer sequences used for quantitative PCR

| | |
|---|---|
| Forward Primer | Reverse Primer (SEQ ID NO) |

| | | | | |
|---|---|---|---|---|
| GLUT1 | ggggcatgattggctccttctctgtg | (SEQ ID NO: 1) | aggccgcagtacacaccgatgatgaa | (SEQ ID NO: 8) |
| GLUT2 | aactttcatttttggtaggcttatca | (SEQ ID NO: 2) | cctcaattaaaaagcaaagcaaacta | (SEQ ID NO: 9) |
| GLUT3 | tgtggtcctatgccgaatgccctcag | (SEQ ID NO: 3) | gcaccaagaagggaaagggagactga | (SEQ ID NO: 10) |
| GLUT4 | cctgcagcctggtagaattgggaagc | (SEQ ID NO: 4) | ttccctccaatcccccttctctagca | (SEQ ID NO: 11) |
| GLUT5 | cccgagtcaattaaacagctggtcct | (SEQ ID NO: 5) | ggaagaccLgttggggccaccgagtt | (SEQ ID NO: 12) |
| GLUT8 | ccctcctLcctgtcatgctccctcca | (SEQ ID NO: 6) | cccgataaggcagccgcgtgagagga | (SEQ ID NO: 13) |
| GLUT9 | ccaaaaacagaacctatgcagaaatc | (SEQ ID NO: 7) | aggccttccatttatcttaccatcag | (SEQ ID NO: 14) |

### Example 2

Glucose Uptake in Xenopus Oocytes

To assess transport function of a specific transporter protein, it can be desirable to clone the cDNA and express the protein in cells that have low endogenous transport activity. Human GLUT3 was cloned by PCR, fully sequenced, and subcloned into plasmids that can be used for expression in mammalian cells or Xenopus oocytes. Because many cell lines already exhibit high levels of GLUT3 activity, expression in Xenopus oocytes can be advantageous due to the low levels of endogenous sugar transport. For expression in Xenopus oocytes, in vitro GLUT3 cRNA was prepared and injected into defoliculated oocytes.

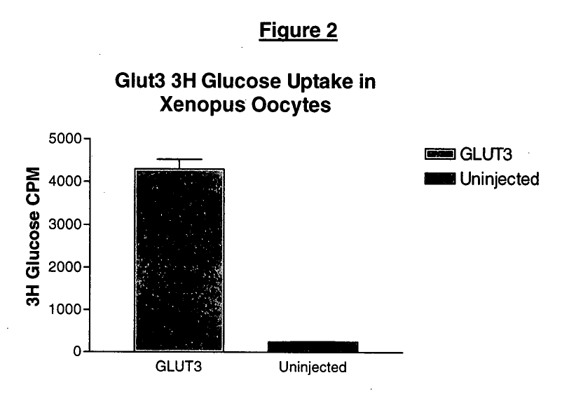

Oocytes expressing GLUT3 exhibited higher levels of ³H-glucose uptake than noninjected controls, as shown in FIG. 2. Oocytes expressing GLUT3 or control oocytes not expressing GLUT3 were incubated in an oocyte ringers (ND96) buffer (90 mM NaCl, 10 mM HemiNa HEPES, 2 mM KCl, 1 mM MgCl, 1.8 mM CaCl₂) containing 0.5% bovine serum albumin and ³H-glucose (10⁶ CPM/ml) for 3 minutes. Oocytes were washed and uptake of radiolabel quantified by scintillation counting.

### Example 3

Glucose Competition Assay in Xenopus Oocytes

To determine the affinity that compounds interact with GLUT3, competition binding assays were performed. The methods used were the same as those in Example 2, except that different concentrations of unlabeled glucose were combined with the ³H-glucose, and the dose response for inhibition of ³H-glucose uptake was measured.

### Example 4

### Glucoronic Acid Uptake in HEK-TREx cells

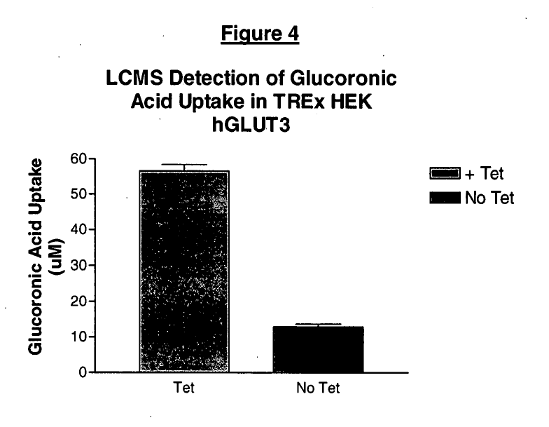

Competition binding studies only demonstrate that a molecule binds the GLUT3 transporter, but do not demonstrate whether the molecule is a substrate and is translocated across the plasma membrane or is a non-transported inhibitor or a non-transported ligand. In order to measure whether test compounds are translocated across the membrane, and to determine the maximal transport rate, a direct uptake method was developed that utilizes mass spectroscopy. For direct uptake measurements using mass spectroscopy, GLUT3-HEK/TREX cells were prepared similarly to those used for competition studies (described above). To measure transport of test compound, GLUT3 expressing HEK cells were washed and incubated with test compounds such as glucuronic acid. Excess test compound was removed by washing with cold assay buffer. Cells were lysed with 50% ethanol/water and the cell debris was pelleted by centrifugation. The supernatant was analyzed by LC-MS-MS. As a negative control, uptake was measured in cells that were not treated with tetracycline. Data from a translocation experiment using glucuronic acid is shown in FIG. 4.

### Example 5

Glucose Competition Assay in HEK-TREx cells

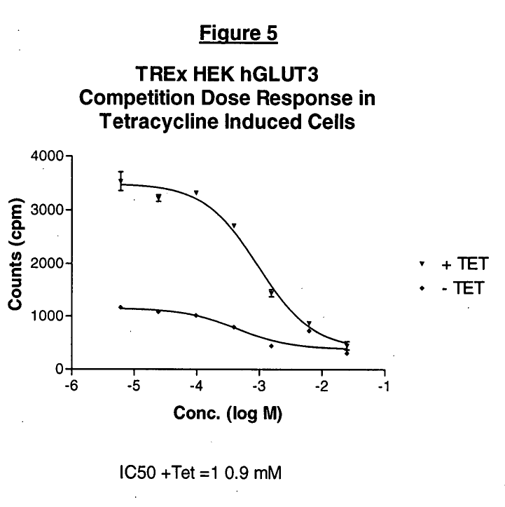

To determine if a test compound binds the GLUT3 transporter, a competition binding assay was developed. This assay measures how different concentrations of a test compound block the uptake of a radiolabeled substrate such as glucose. The half-maximal inhibitory concentration (IC₅₀) for inhibition of transport of a substrate by a test compound is an indication of the affinity of the test compound for the GLUT3 transporter. If the test compound binds GLUT3 competitively with the radiolabeled substrate, less of the radiolabeled substrate is transported into the HEK cells. For test compounds do not interact with GLUT3 in a manner competitive with substrates the curve remains an essentially flat line (not shown in FIG. 5), i.e., there is no dose response seen. The amount of radiolabeled substrate which is taken up by the cells is measured by lysing the cells and measuring the radioactive counts per minute.

Competition binding studies were performed as follows. GLUT3-HEK/TREX cells were plated in 96-well plates at 100,000 cells/well at 37° C. for 24 hours and tetracycline (1 μg/mL) was added to each well for an additional 24 hours to induce GLUT3 transporter expression. Radiolabeled ³H-glucose (˜75,000 cpm/well) was added to each well in the presence and absence of various concentrations of unlabeled glucose in duplicate or triplicate. Plates were incubated at room temperature for 1 min. Excess ³H-glucose was removed and cells were washed three times with a 96-well plate washer with cold assay buffer. Scintillation fluid was added to each well, and the plates were sealed and counted in a 96-well plate-based scintillation counter. Data was graphed and analyzed using non-linear regression analysis with Prism Software (GraphPad, Inc., San Diego, Calif.). An example of results from the assay is shown in FIG. 5.

### Example 6

Efflux Assay Using Reconstituted Membranes

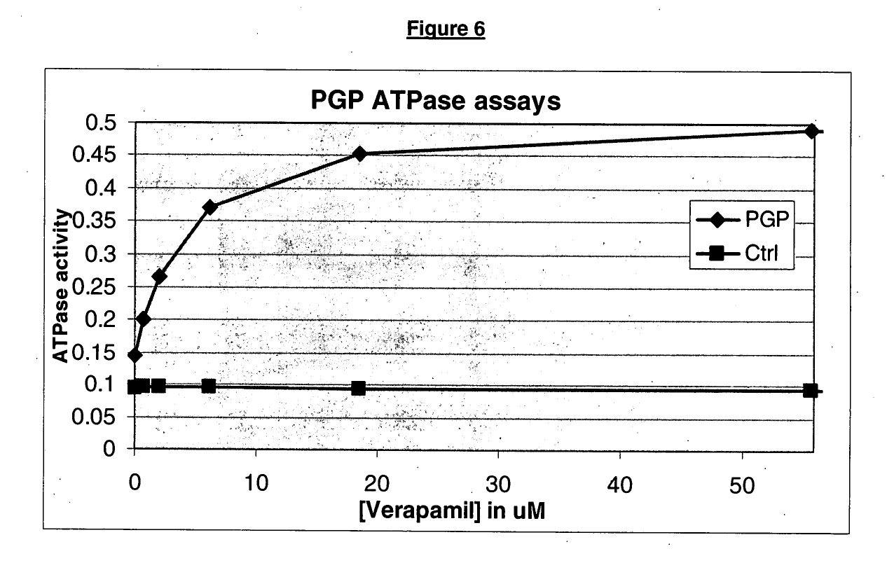

FIG. 6 depicts the results of an efflux experiment in which the PgP substrate verapamil was added to commercial Baculovirus membranes (purchased from BD Biosciences) at various concentrations depicted on the X axis followed by ATPase activity measurement. The ATPase activity measurement was performed using the lactate dehydrogenase/pyruvate kinase coupled enzyme system described by Tietz & Ochoa, Arch. Biochim. Biophys. Acta 78:477 (1958) to follow the decrease in absorbance at 340nm resulting from the oxidation of NADH, which is proportional to ATPase activity. SmM sodium azide (NaN₃), 1 mM EGTA, and 0.5 mM Ouabain, each of which inhibit non-specific ATPases in the membranes, were added to the reactions to further enhance the specificity of the PgP ATPase signal. The other components in the assay mixture were 25 mM Tris, pH 7.8, 100 mM NaCl, 10 mM KCl, 5 mM MgCl₂, 1 mM DTT, 2 mM phosphoenolpyruvate, 1 mM NADH, 0.1 mg/ml lactate dehydrogenase, 0.1 mg/ml pyruvate kinase, 5 mM ATP, and 6 ug PgP or control membranes. FIG. 6 demonstrates that as the concentration of verapamil was increased, the ATPase activity in PgP-containing membranes but not in control membranes also increased.

### Example 7

Efflux Competition Assay

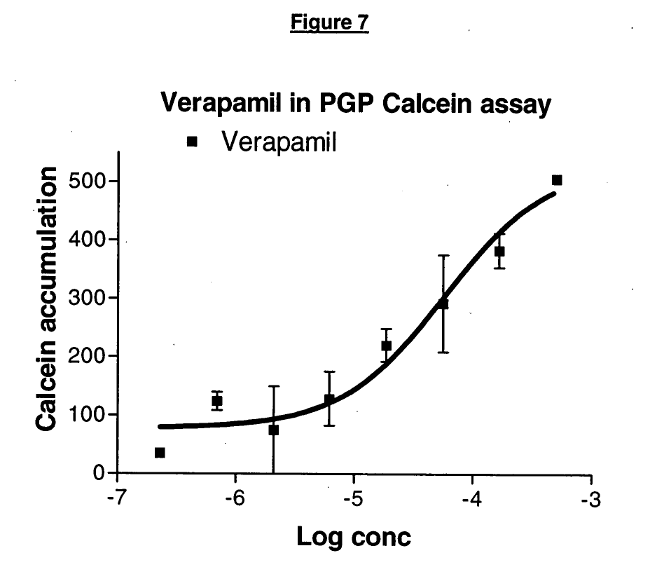

FIG. 7 depicts the results of an efflux competition assay. A tetracycline-inducible PgP expression construct (TREx-PgP) was transfected into HEK cells. The cells were incubated with PgP substrate 5 μM calcein-AM, which passively diffuses into the cells, as well as with various concentrations of the PgP substrate verapamil as. shown in FIG. 7. As the concentration of PgP substrate verapamil was increased, more calcein-AM accumulated in the cells and was converted to the fluorescent product calcein.

### Example 8

Staining of Tumor Samples

Immunohistochemical staining of tumor tissue microarrays enables the expression patterns of the GLUT3 transporter within tumor tissues to be examined. As a first step, developing antibodies that bind to the GLUT3 transporter were developed and stained against a panel of human tumor samples. The results are summarized in Table 5.

A unique, relatively hydrophilic, stretch of amino acids (TRAFEGQAHGADRSGKDGVMEMNSIEPAKETTNV) (SEQ ID NO: 15) was identified for the GLUT3 transporter using Vector NTI and BLAST analysis. Using PCR, this region of the transporter was amplified from cDNA using primers containing Bam-HI and EcoRI restriction sites to allow directional cloning into the GST-fusion vector pGEX-6P-1 (Amersham Biosciences). Constructs were sequenced and then placed into an IPTG inducible bacterial system to overexpress the GST-fusion protein. The protein was affinity purified and sent to CoCalico Biologicals, Inc. for polyclonal antibody production.

Cos-7 cells were transiently transfected with the indicated transporter or left untransfected as a mock control. Whole cell lysates were made, and Western Blot analysis was performed using the indicated affinity purified polyclonal antibody. The antibodies are specific, and upon transfection, there was an increased signal of a protein band of the expected size. Some cross-reactivity with endogenous monkey transporter was observed.

Commercially available tumor tissue microarrays (Ambion) were used having the following characteristics: large sample size (50-250 tissues) per slide, matched benign controls, multiple types of tumors present on each slide, and having clinical annotations for the various tissues.

The following staining procedure was used. Paraffin slides purchased from Ambion were baked for 1 hr at 37° C. and then for thirty minutes at 55° C. Tissues were then dewaxed with Biogenex EZ-DeWax solution as instructed by the manufacturer. Dewaxed slides were placed in an antigen retriever containing Retrievit Solution pH 8.0 (BioGenex). After briefly rinsing with water, tissues were blocked for endogenous peroxides with 3% hydrogen peroxide for 10 minutes. Slides were then rinsed with water and blocked with avidin followed by biotin for 15 minutes each. Non-specific binding was blocked by incubation in SuperBlock (PIERCE)+0.5% normal goat serum for 1 hr. Slides were then incubated with primary antibody diluted in block for 1.5 hr. Specimens were then rinsed three times for 5 minutes with PBS+0.1% Tween 20. Tissues were then incubated with biotinylated goat anti-rabbit immunoglobulins for 20 minutes, rinsed as above, and then incubated with streptavidin-horseradish peroxidase for 20 minutes. Slides were developed using DAB (diaminobenzidine) and hematoxylin as a nuclear counterstain. Tissues were covered with SuperMount (BioGenex) and then air dried. The slides were examined under the microscope and scored for intensity of staining using a scale of zero to four (0 to 4), with a score of zero being the lightest staining (i.e., a staining that was similar to the staining achieved in the negative controls) and a score of four being the most heavily stained. Numbers in table 5 are percentage transporter expression equal to or greater than 3 on a scale of 1-4 in various cancers.

TABLE 5

| | GLUT3 |
|---|---|
| BRAIN CANCER | |
| Astrocytoma (10) | 15% |
| Glioblastoma multiforme (28) | 18% |
| Normal Brain (11) | 0% |
| LUNG CANCER | |

1. A method of screening an agent, conjugate or conjugate moiety for activity useful for treating or diagnosing cancer, comprising:
(a) providing a cell expressing a GLUT3 transporter, the transporter being situated in the plasma membrane of the cell;
(b) contacting the cell with an agent, conjugate or conjugate moiety; and
(c) determining whether the agent, conjugate or conjugate moiety passes through the plasma membrane via the GLUT3 transporter, passage through the GLUT3 transporter being useful for treatment or diagnosis of cancer;
wherein:
if step (b) comprises contacting the cell with the agent, the agent is a cytotoxic agent or an imaging component;
if step (b) comprises contacting the cell with the conjugate, the conjugate comprises an agent that is a cytotoxic agent or an imaging component; or
if step (b) comprises contacting the cells with the conjugate moiety, the method further comprises linking the conjugate moiety to an agent that is a cytotoxic agent or an imaging component.

2. The method of claim 1, further comprising:
(d) contacting the agent, conjugate, or conjugate moiety, with a cancerous cell and determining whether the agent kills or inhibits growth of the cell.

3. The method of claim 2, wherein the cancerous cell is present in an animal.

4. The method of claim 1, wherein:
(i) the cell endogenously expresses the GLUT3 transporter; or
(ii) a nucleic acid molecule encoding the GLUT3 transporter has been transfected or injected into the cell.

5. The method of claim 4, wherein the cell is a human cancer cell that has not been genetically manipulated.

6. The method of claim 4, wherein the cell is an oocyte.

7. The method of claim 4, wherein the cell is a human embryonic kidney (HEK) cell.

8. The method of claim 4, wherein the determining is performed by a competition assay.

9. The method of claim 4, wherein the determining is performed by a direct uptake assay.

10. The method of claim 1, further comprising:
(d) administering the agent, conjugate, or conjugate moiety to an animal and measuring the amount of agent, conjugate, or conjugate moiety that is taken up by cancerous cells in the animal.

11. The method of claim 1, wherein the determining step determines that the agent, conjugate or conjugate moiety passes through the plasma membrane via the GLUT3 transporter and the method further comprises:
(d) modifying the agent, conjugate or conjugate moiety; and
(e) determining if the modified agent, conjugate or conjugate moiety is transported with a higher Vₘₐₓ by the GLUT3 transporter than the agent, conjugate or conjugate moiety.

12. The method of claim 1, wherein the cytotoxic agent is selected from the group consisting of platinum, nitrosourea, a phoshoramide group that is selectively cytotoxic to brain tumor cells, nitroimidizole, and nitrogen mustard.

13. The method of claim 1, wherein the agent, conjugate or conjugate moiety comprises at least one 5 or 6 membered ring.

14. The method of claim 13, wherein the agent, conjugate or conjugate moiety is selected from the list consisting of glucose, glucorionic acid, dehydroascorbic acid, glucosamine, and fluorodeoxyglucose.

15. The method of claim 1, further comprising administering the agent, conjugate or conjugate moiety to an undiseased animal and determining any toxic effects.

16. The method of claim 1, further comprising:
(d) determining that the agent, conjugate or conjugate moiety is transported by at least one efflux transporter.

17. The method of claim 16, further comprising:
(e) modifying the agent, conjugate or conjugate moiety;
(f) establishing that the modified agent, conjugate or conjugate moiety retains GLUT3 substrate activity; and
(g) comparing the ratio of GLUT3 substrate activity to the ratio of efflux substrate activity for the agent, conjugate or conjugate moiety and the modified agent, conjugate or conjugate moiety wherein an increased ratio of GLUT3 substrate activity to efflux substrate activity demonstrates that the modification improves the usefulness of the agent, conjugate or conjugate moiety for treatment or diagnosis of cancer.

18. The method of claim 17, wherein the efflux substrate activity is determined by conducting an assay selected from the group consisting of:
(i) an efflux transporter ATPase activity assay; and
(ii) an efflux transporter competition assay.

19. A conjugate comprising a cytotoxic agent or imaging component which is transported into cancer cells, identified by the method of claim 10.

20. A pharmaceutical composition comprising a cytotoxic agent or an imaging component linked to a conjugate moiety to form a conjugate, wherein the conjugate has a higher Vₘₐₓ for GLUT3 than the cytotoxic agent or the imaging component alone.

21. The pharmaceutical composition of claim 20, wherein the conjugate has at least 5 times the Vₘₐₓ for GLUT3 than the cytotoxic agent or the imaging component alone.

22. The pharmaceutical composition of claim 20, wherein the conjugate has a lower Vₘₐₓ for an efflux transporter than the cytotoxic agent or the imaging component alone.

23. The pharmaceutical composition of claim 20, wherein the conjugate moiety has a Vₘₐₓ for GLUT3 that is at least about 1% of the Vₘₐₓ of glucose for GLUT3.

24. The pharmaceutical composition of claim 20, wherein the conjugate has a Vₘₐₓ for GLUT3 that is at least 5% of the Vₘₐₓ of glucose for GLUT3.

25. The pharmaceutical composition of claim 20, wherein the conjugate moiety has a Vₘₐₓ for GLUT3 that is at least about 50% of the Vₘₐₓ of glucose for GLUT3.

26. A method of formulating a conjugate, comprising:
(a) linking a cytotoxic agent or imaging component to a conjugate moiety to form the conjugate, wherein the conjugate has a greater Vₘₐₓ for a GLUT3 transporter than the cytotoxic agent or imaging component alone; and
(b) formulating the conjugate with a pharmaceutical carrier as a pharmaceutical composition.

27. A method of delivering a conjugate, comprising administering to a patient a pharmaceutical composition comprising a cytotoxic agent or imaging component linked to a conjugate moiety to form the conjugate, wherein the conjugate has a higher Vₘₐₓ for a GLUT3 transporter than the cytotoxic agent or imaging component alone, and wherein the conjugate is transported into cancerous cells of the patient.

28. The method of claim 27, wherein the Vₘₐₓ of the conjugate is at least two-fold higher than that of the cytotoxic agent or imaging component alone.

29. The method of claim 27, wherein the cytotoxic agent is selected from the group consisting of platinum, nitrosourea, a phosphoramide group selectively cytotoxic to brain tumor cells, nitroimidizole, and nitrogen mustard.

30. The method of claim 27, wherein the cancerous cells are present in a solid tumor.

31. The method of claim 27, further comprising determining a level of expression of GLUT3 in the cancerous cells in excess of a level in noncancerous cells from the same tissue.

32. The method of claim 27, wherein the cytotoxic agent is a nitroimidizole and the method further comprises irradiating the patient to kill cancerous cells that have taken up the conjugate.

33. A method of screening an agent for pharmacological activity useful for treating cancer, comprising:
(a) determining whether an agent binds to a GLUT3 transporter; and
(b) contacting the agent with a cancerous cell and determining whether the agent kills or inhibits growth of the cell, killing or inhibition of growth indicating the agent has the pharmacological activity.

34. The method of claim 33, further comprising
(c) contacting a cell expressing a GLUT3 transporter with a substrate of the GLUT3 transporter, and determining whether the agent inhibits uptake of the substrate into the cancerous cell.

35. The method of claim 33, wherein the cell is a HEK cell.

36. The method of claim 33, wherein the substrate is glucose.

37. The method of claim 33, further comprising administering the agent to an undiseased animal and determining any toxic effects.
